(19) United States
(12) Patent Application Publication
(10) Pub. No.: US 2025/0176877 A1
(43) Pub. Date: Jun. 5, 2025

(54) SENSING PLATFORMS WITH NEEDLES AND METHODS OF FABRICATION AND USE

(71) Applicant: Purdue Research Foundation, West Lafayette, IN (US)

(72) Inventors: Chi Hwan Lee, West Lafayette, IN (US); Dong Rip Kim, Seoul (KR); Hyunjoon Kong, Champaign, IL (US); Woohyun Park, Fremont, CA (US)

(21) Appl. No.: 18/965,670

(22) Filed: Dec. 2, 2024

Related U.S. Application Data
(60) Provisional application No. 63/605,343, filed on Dec. 1, 2023.

(51) Int. Cl. A61B 5/1473; A61B 5/00

(52) U.S. Cl. CPC A61B 5/1473; A61B 5/685; A61B 2562/0285; A61B 2562/164

(57) ABSTRACT

Sensing platforms having micro-sized needles on a flexible substrate, and methods for their fabrication and use. Such a sensing platform includes a flexible substrate, micro-sized needles extending from a surface of the flexible substrate, a percolated network of electrically-conductive nanowires on the surface of the flexible substrate; and an electrically-insulating layer disposed on the surface of the flexible substrate and covering the percolated network of electrically-conductive nanowires so that only tips of the micro-sized needles protrude from and are exposed by the electrically-insulating layer.

### CROSS-REFERENCE TO RELATED APPLICATIONS

This application claims the benefit of U.S. Provisional Application No. 63/605,343 filed Dec. 1, 2023, the contents of which are incorporated herein by reference.

### STATEMENT REGARDING FEDERALLY SPONSORED RESEARCH OR DEVELOPMENT

This invention was made with government support under CBET 2032529 awarded by the National Science Foundation. The government has certain rights in the invention.

### BACKGROUND OF THE INVENTION

The present invention generally relates to sensing devices. The invention particularly relates to sensing platforms that comprise micro-sized needles on a flexible substrate and are capable of use in intracellular recordings.

Intracellular recording is pivotal in deepening the understanding of cellular function, promoting therapeutic development, advancing drug discovery, and stimulating technological innovation in a range of fields, including neuroscience, cardiology, muscle physiology, and drug screening. Patch-clamp techniques remain the current standard for intracellular recording, providing high-fidelity recordings of cellular activity. However, the invasive nature of patch-clamp techniques limits their widespread applications in many fields. An alternative approach involves utilizing vertically-ordered one-dimensional (1D) nanostructures, such as wires, needles, pillars, straws, and/or tubes, at cellular and subcellular length scales to serve as bioprobes with high spatial resolution. Compared to other options, Si needles are of particular interest for intracellular recording due to their minimal invasiveness and ability to infiltrate living cells with negligible damage, low toxicity to cells, and compatibility with traditional nanofabrication processes.

Real-time live imaging, often combined with the direct recording of electrical signals from electrodes, is a crucial component of intracellular recording, enabling the visualization of cellular and subcellular morphology with a high level of specificity. In addition to providing valuable insights into the location and behavior of recorded cells, live imaging offers a comprehensive understanding of the underlying cellular processes being studied. Access to this information enables researchers to gain a deeper understanding of the intricate physiological functions and signaling pathways of cells, thereby enhancing the precision and dependability of intracellular recording. However, standard live imaging techniques, such as inverted and confocal microscopes, require inverting the device or using planar micro-electrode arrays on transparent substrates, which are typically limited to recording extracellular signals.

Though of particular interest for long-term intracellular recording owing to their capacity to infiltrate living cells with negligible damage and minimal toxicity, vertically ordered Si needles have yet to be implemented due to the opacity of Si wafers typically employed for fabricating vertical Si needles. Furthermore, the rigidity of Si wafers may cause mechanical discrepancies with cells during intracellular recordings, leading to cell stress and damage that can impair the quality of intracellular signals. Therefore, it is necessary to integrate vertical Si needles onto a transparent and flexible substrate, thereby facilitating simultaneous intracellular recording and imaging with minimal invasiveness.

It would be desirable if improved methods were available for fabricating and transferring Si needles onto flexible substrates.

### BRIEF SUMMARY OF THE INVENTION

The intent of this section of the specification is to briefly indicate the nature and substance of the invention, as opposed to an exhaustive statement of all subject matter and aspects of the invention. Therefore, while this section identifies subject matter recited in the claims, additional subject matter and aspects relating to the invention are set forth in other sections of the specification, particularly the detailed description, as well as any drawings.

The present invention provides, but is not limited to, sensing platforms having micro-sized needles on a flexible substrate, and to methods for their fabrication and use.

According to a nonlimiting aspect of the invention, a sensing platform includes a flexible substrate, micro-sized needles extending from a surface of the flexible substrate, a percolated network of electrically-conductive nanowires on the surface of the flexible substrate; and an electrically-insulating layer disposed on the surface of the flexible substrate and covering the percolated network of electrically-conductive nanowires so that only tips of the micro-sized needles protrude from and are exposed by the electrically-insulating layer.

According to another nonlimiting aspect of the invention, a method of using a sensing platform as described above includes depositing a composition within the cell culture well so as to contact and be pierced by the tips of the micro-sized needles extend; and then causing the sensing platform to electrically interact with the composition.

According to another nonlimiting aspect of the invention, a method of fabricating a sensing platform as described above includes providing a first substrate having pillars extending from a surface thereof, locally reducing diameters of the pillars at locations thereof adjacent the first substrate, embedding distal ends of the pillars in the flexible substrate, expanding the flexible substrate to cause the pillars to fracture at the locations thereof adjacent the first substrate and detach therefrom to define the micro-sized needles extending from the flexible substrate, depositing the percolated network of electrically-conductive nanowires on the surface of the flexible substrate, and then depositing the electrically-insulating layer on the surface of the flexible substrate so as to cover the percolated network of electrically-conductive nanowires and so that only the tips of the micro-sized needles protrude from and are exposed by the electrically-insulating layer.

Technical aspects of sensing platforms and methods having features as described above preferably include the ability to integrate vertical Si needles onto flexible substrates, as a nonlimiting example, for the purpose of producing an optically transparent intracellular sensing platform that can be used to facilitate simultaneous intracellular recording and imaging with minimal invasiveness.

Other aspects and advantages will be appreciated from the following detailed description as well as any drawings.

### BRIEF DESCRIPTION OF THE SEVERAL VIEWS OF THE DRAWING

The patent or application file contains at least one drawing executed in color. Copies of this patent or patent application publication with color drawing(s) will be provided by the Office upon request and payment of the necessary fee.

FIGS. 1A through 1D contain schematic illustrations (top panel of each figure) and scanning electron microscope (SEM) images (bottom panel of each figure) detailing the fabrication of a sensing platform as a nonlimiting example of the present invention, including: (FIG. 1A) the assembly of vertical Si needles on a polydimethylsiloxane (PDMS) flexible substrate and a polyethylene terephthalate (PET) film, (FIG. 1B) the application of Ag—Au nanowires (NW) via spray coating to enhance conductivity, (FIG. 1C) insulation and tip exposure achieved through PDMS spin-coating and plasma etching, and (FIG. 1D) the cultivation of an HL-1 cell monolayer on the vertical Si needles.

FIG. 2A contains finite element analysis (FEA) results displaying the strain distribution (E) and deflection (6) of a single vertical Si needle on a PDMS substrate with Ag—Au nanowires, under a contraction force (F) of 5 μN exerted by a cell. FIG. 2B contains FEA results illustrating deflection (6) of the vertical Si needle, considering varying applied forces and exposed lengths (L). FIG. 2C is a graph plotting optical transmittance measurements for various configurations including a bare PDMS flexible substrate, vertical Si needles on the PDMS flexible substrate, Ag—Au nanowires on the PDMS flexible substrate, and a sensing platform such as represented in FIGS. 1A-1D. FIG. 2D depicts differential interference contrast (DIC), confocal, and merged images showing an HL-1 cell on the sensing platform. FIG. 2E is a graph plotting changes in electrochemical impedance in the sensing platform relative to controls, measured at 1 kHz over time, during immersion in phosphate buffer solution (PBS) and incubation at 37° C. with 5% CO₂. Data is represented as the mean±standard deviation, with n=5 for each group. FIG. 2F is a bar graph presenting cell viability results for the sensing platform in comparison to controls, depicted as the mean±standard deviation, with n=5 for each group.

FIG. 3A contains graphs plotting recordings of HL-1 action potential, showing extracellular signals (blue) prior to electroporation and intracellular signals (red) following electroporation, along with the simultaneous recording of calcium flux measurement (green). FIG. 3B contains images from a video and capture changes in HL-1 calcium flux during a single action potential. FIG. 3C is a graph evidencing effects of the ion channel-blocking drug nifedipine. FIG. 3D contains images of calcium flux, showing baseline and peak levels before and after treatment with nifedipine. FIG. 3E contains 3D (top) and cross-sectional (bottom) images, labeled with cell membrane (red) and vertical Si needles (blue), to demonstrate the tight interface created between the vertical Si needles and the cell membrane. FIG. 3F depicts a DIC image merged with confocal imaging and shows the vertical Si needles (gray) overlapped with membrane (red) and nucleus (blue).

FIG. 4A contains graphs plotting intraorganoid field potential measurement (red) along with a simultaneous recording of calcium flux measurement (green). FIG. 4B contains fluorescence images of calcium flux within cells from cardiovascular organoids. FIG. 4C contains fluorescence images that reveal the expression of cTnT and α-actinin in cardiovascular organoids. FIG. 4D is a bar graph evidencing relative gene expression of cardiovascular and calcium handling markers in cells from cardiovascular organoids, normalized to cells from embryonic bodies, with n=10 for each group. FIG. 4E contains 3D images of a cardiovascular organoid interfacing with the vertical Si needles, stained for actin (red), nucleus (blue), and vertical Si needles (green). FIG. 4F contains cross-sectional (top) and 3D (bottom) images of the vertical Si needles placed into the cardiovascular organoid, with staining for actin (red), nucleus (blue), and vertical Si needles (green). A white dotted circle illustrates the flexible nature of the vertical Si needles, showing how they are buckled towards the organoid.

FIGS. 5A through 5F schematically represent a series of steps performed according to a nonlimiting example of fabricating the vertical Si needles on a Si wafer and transfer printing onto a PDMS flexible substrate.

### DETAILED DESCRIPTION OF THE INVENTION

The intended purpose of the following detailed description of the invention and the phraseology and terminology employed therein is to describe what is shown in the drawings, which include the depiction of and/or relate to one or more nonlimiting embodiments of the invention, and to describe certain but not all aspects of what is depicted in the drawings. The following detailed description also describes certain investigations relating to the embodiment(s) depicted in the drawings. Therefore, the appended claims, and not the detailed description, are intended to particularly point out subject matter regarded to be aspects of the invention, including certain but not necessarily all of the aspects and alternatives described in the detailed description.

The following disclosure describes various aspects of needles integrated onto a flexible substrate, as a nonlimiting example, for the purpose of combining vertical Si needles on an optically transparent flexible substrate to yield an optically transparent intracellular sensing platform that can be used to facilitate simultaneous intracellular recording and imaging with minimal invasiveness. Such sensing platforms preferably include a percolated network of electrically conductive nanowires that are covered by an electrically-insulating layer applied to the surface of the flexible substrate to expose only limited portions of the tips of the needles that protrude from the electrically-insulating layer. Such sensing platforms are preferably capable of meeting prerequisites for simultaneous intracellular recording and imaging, encompassing electrochemical impedance, optical transparency, mechanical compliance, and cell viability. Investigations leading to the present invention demonstrated that sensing platforms as described above are capable of exhibiting desirable characteristics including: a low electrochemical impedance, measuring less than 110 Ω cm² at 1 kHz, which remained stable for over 28 days of intracellular recording; excellent optical transparency exceeding 80% in the 400-700 nm range, facilitating standard live imaging techniques such as inverted and confocal microscopes; mechanical compliance that permits the Si needles to interface with cells without the risk of fracture or delamination from the substrate; and outstanding cell viability, exceeding 99%. The investigations further demonstrated the ability of the sensing platform to monitor electrical potentials in different types of cells, such as cardiomyocytes and three-dimensionally (3D) engineered cardiovascular tissue, while simultaneously conducting live imaging with inverted and confocal microscopes. These demonstrations included recording intracellular electrophysiology during live imaging before and after the administration of therapeutic drugs. As such, the sensing platform is believed to have wide-ranging potential applications for intracellular research across various disciplines, such as neuroscience, cardiology, muscle physiology, and drug screening, and have the potential to advance drug screening and therapies for cardiovascular or other diseases by facilitating comprehensive electrical and optical monitoring of cellular activity over time.

To facilitate the description provided below of embodiments represented in the drawings, relative terms, including but not limited to, “proximal,” “distal,” “vertical,” “horizontal,” “lateral,” “front,” “rear,” “side,” “forward,” “rearward,” “top,” “bottom,” “upper,” “lower,” “above,” “below,” “right,” “left,” etc., may be used in reference to the orientation of the sensing platforms as represented in the drawings. All such relative terms are useful to describe what is shown in the drawings, but should not be otherwise interpreted as limiting the scope of the invention.

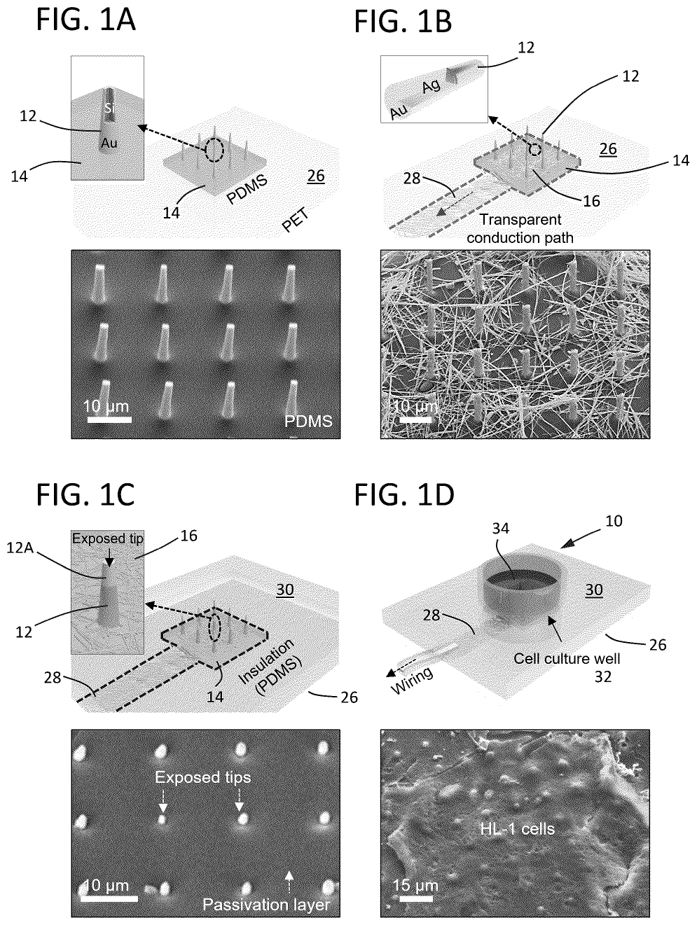

FIGS. 1A through 1D represent a process of fabricating a representative but nonlimiting transparent intracellular sensing platform 10 that combined vertical Si needles 12 with a percolated network of electrically-conductive nanowires 16 on an optically transparent flexible (e.g., an elastomer such as PDMS) substrate 14 during investigations leading to the present invention. While certain materials are indicated below and were used during investigations leading to the present invention, those skilled in the art will be aware of alternative materials that could be used and such materials are within the scope of the invention.

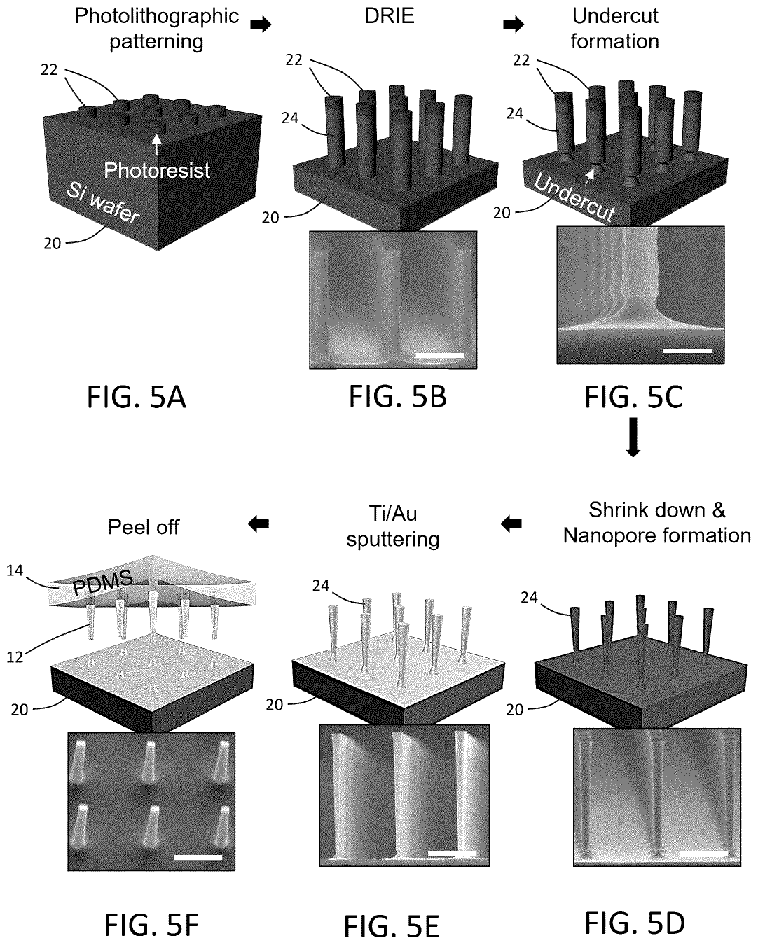

Fabrication was initiated with a silicon wafer with which the vertically ordered Si needles 12 are created through photolithographic patterning and deep reactive ion etching (DRIE). This process as employed during the investigations is schematically represented in FIGS. 5A through 5D, which show islands of photoresist 22 photolithographically patterned on a silicon wafer 20 (FIG. 5A), DRIE etching the wafer 20 to form micro-sized (dimensions of less than 1000 m) silicon pillars 24 (FIG. 5B), and undercutting the pillars 24 at their bases adjacent the resulting etched surface of the wafer 20 (FIG. 5C). To undercut the Si pillars 24, additional isotropic etching was conducted using SF₆ gas at a flow rate of 85 sccm, 450 W of RF plasma power, and 30 W of platen power. To ensure minimal invasiveness when the Si needles 12 are introduced into cells, the pillars 24 were tapered (FIG. 5D) from a base diameter of less than 2 μm at the distal ends of the pillars 24 (corresponding to the bases of the needles 12 in FIG. 5F) to a diameter of less than 900 nm adjacent the wafer 20 (corresponding to the tips of the needles 12 in FIG. 5F). Tapering was performed by immersion in a 15 wt. % potassium hydroxide (KOH) solution at 25° C. The surface areas of the pillars 24 were then increased to decrease impedance of the eventual Si needles 12 by texturing the surfaces of the pillars 24 with nanopores. A metal-assisted chemical etching (MACE) process was employed for this purpose, which involved immersing the specimen in an etching solution made up of 20 mM Ag nitrate (AgNO₃) and 49% hydrofluoric (HF) solution. After the MACE process, any Ag residues on the surfaces of the pillars were removed by immersing in an Ag etchant solution. The resulting nanopores had diameters ranging from 5 to 20 nm, which led to an overall porosity of 30% in the needles 12. The pillars 24 were then coated by sputtering a thin film of Ti/Au (3 nm/50 nm) (FIG. 5E).

Thereafter, the pillars 24 were transfer printed onto the flexible substrate 14 (identified in FIG. 5F as polydimethylsiloxane (PDMS)) and separated from the wafer 20 by breaking the pillars 24 at their undercut bases to yield the needles 12 (FIG. 5F). To initiate the process, an uncured PDMS solution in a 10:1 weight ratio of the base polymer and curing agent was spin-cast onto the substrate 14 (pre-cured PDMS). The PDMS solution was partially cured at room temperature to serve as an adhesive layer. The pillars 24 were then positioned upside down on the PDMS adhesive. A subsequent annealing process was conducted to complete the polymerization of the PDMS adhesive and ensure the physical bonding of the needles 12 to the flexible substrate 14. To release the needles 12 from the Si wafer 20, the entire specimen was immersed in a solvent that caused the PDMS flexible substrate 14 to swell, leading to the controlled cracking of the bottom undercuts of the pillars 24 where mechanical stress can be concentrated during the swelling process. Finally, the resulting specimen was washed with deionized (DI) water and dried in a convection oven at 70° C. for 1 hour to remove any remaining solvent, enabling the substrate 14 to recover to its original size.

FIGS. 1A through 1D contain schematic illustrations (top panels) and scanning electron microscope (SEM) images (bottom panels) of the subsequent fabrication processes that were used in the investigations to complete the fabrication of the sensing platform 10 from the flexible substrate 14 and its needles 12 depicted in FIG. 5F. In FIG. 1A, the substrate 12 is represented as bonded to a polyethylene terephthalate (PET) film 26 to serve as a handling carrier. In FIG. 1B, gold-coated silver (Au—Ag) nanowires 16 were prepared and uniformly sprayed across the surface of the substrate 14 with a shadow mask to define a percolated network of the Au—Ag nanowires 16 on the substrate 14. The percolated network of nanowires 16 are also shown as deposited on the film 26 to define an optically transparent conduction path 28 that leads from the substrate 14 to, for example, a connection pad (not shown). The SEM image in FIG. 1B highlights the percolated network of Au—Ag nanowires 16 in contact with the needles 12, establishing an electrical connection therebetween. The surfaces of the Au—Ag nanowires 16 were treated with (3-aminopropyl) triethoxysilane (APTES) prior to spraying to improve adhesion, and the nanowires 16 were chemically welded after spraying to reduce sheet resistance to 36.81±0.93 Ω sq⁻¹.

FIG. 1C represents the nanowires 16 as preferably covered by an electrically-insulating layer 30 applied to the surface of the flexible substrate 14 to expose only limited portions of the needles 12 that protrude from the electrically-insulating layer 30 as tips 12A of the needles 12. The electrically-insulating layer 30 preferably provides elastic structural support to the needles 12 and fully passivates the Au—Ag nanowires 16 for electrical insulation. As represented in FIG. 1C, the electrically-insulating layer 30 may be formed by a diluted PDMS solution that is spin-cast over the surface of the substrate 14, followed by a reactive ion etching (RIE) process to expose the tips 12A of the needles 12 above the etched surface of the electrically-insulating layer 30. The SEM image in FIG. 1C highlights the electrically-insulating layer 30 with the exposed tips 12A of the needles 12. The lengths of the exposed tips 12A were uniform and controlled by adjusting the RIE time in a range of approximately 2 μm to 25 μm.

In FIG. 1D, a cell culture well 32 (identified as PDMS) is depicted as affixed to the electrically-insulating layer 30 and surrounding the exposed tips 12A of the needles 12A, yielding the sensing platform 10 and enabling the sensing platform 10 to electrically interact with a composition, such as a cell culture, through the needles 12 and percolated network of nanowires 16. The SEM image in FIG. 1D displays a confluent layer 34 of mouse cardiomyocyte (HL-1) cells interfacing with the exposed needle tips 12A after three weeks of cell culture in a phosphate buffer solution (PBS) at 37° C.

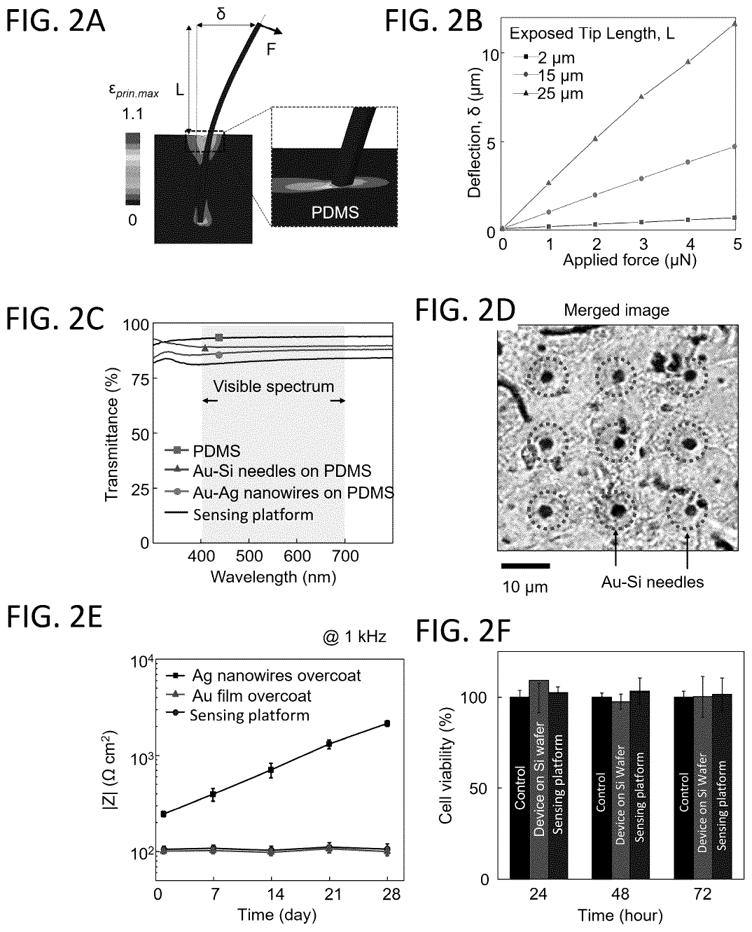

Sensing platforms 10 manufactured as described above were shown to be mechanically compliant, electrically conductive, and optically transparent, which are critical for simultaneous intracellular recording and imaging. FIG. 2A presents the finite element analysis (FEA) results of strain distribution (ε) and deflection (δ) for a single needle 12 extending from a PDMS substrate 14 and interacting with a cell under a contraction force (F) of 5 μN. This testing was performed on multiple needles 12 having tips 12A with different exposed lengths (L) of 2, 15, and 25 μm. The peak principal strains appeared on the top surface of the PDMS substrate 14 surrounding the needles 12, opposite to the direction of the applied force. Consistent results were obtained as the applied force increased up to 5 μN, and the exposed tip lengths (L) of the needles 12 increased from 2 μm to 15 μm and then to 25 μm, with a corresponding increase in deflection (FIG. 2B). The spring constant of the needles 12 was determined to be 1.86 N m⁻¹, 0.35 N m⁻¹, and 0.14 N m⁻¹ for L=2 μm, 15 μm, and 25 μm, respectively. The embedded length of the needles 12 into the PDMS substrate 14 had the ability to rotate, rather than just bend, due to elastic compliance at the interfaces therebetween, which could substantially reduce the strain applied to the needles 12 as compared to that built on a rigid substrate such as a silicon wafer. The needles 12 on the PDMS substrate 14 experienced a maximum Mises stress of less than 1.2 GPa, which is lower than that of the same needle on a Si wafer (approximately 2 GPa). These findings indicated that the needles 12 on a flexible substrate 14 may help reduce the risk of fractures or delamination during cell interactions, a conclusion that is in line with subsequent experimental observations.

FIG. 2C displays the optical transmittance of the sensing platform 10 in the visible spectrum (400-700 nm), which measures greater than 80% compared to the control samples, including a bare PDMS substrate 14, the needles 12 on a PDMS substrate 14, and a percolated network of the Au—Ag nanowires 16 on a PDMS substrate 14. The density of the Au—Ag nanowires 16 was increased by spraying them for durations ranging from 5 to 30 seconds, resulting in a slight decrease in optical transmittance and an exponential decrease in sheet resistance. To optimize both optical transmittance and sheet resistance of the sensing platform 10, the Au—Ag nanowires 16 were sprayed for 15 seconds, resulting in an optical transmittance of greater than 80% and a sheet resistance of less than 100 Ω sq⁻¹. FIG. 2D presents a merged image of differential interference contrast (DIC) and confocal microscope of HL-1 cells in conjunction with the needles 12, revealing targeted cellular structures such as cell membranes and nuclei. Inverted and confocal microscopes were utilized to obtain these real-time, label-free images, which are feasible only with the presence of the optically transparent substrate 14.

FIG. 2E shows the electrochemical impedance of the sensing platform 10 immersed in PBS at a frequency of 1 kHz over a 28-day period, while incubated at 37° C. and 5% carbon dioxide (CO₂). For comparison, control groups included substantially identical Si needles coated with a 50 nm-thick Au film applied via sputtering and a percolated network of substantially identical Ag nanowires, both set on a Si wafer. The results revealed that the sensing platform 10, as well as the control device coated with the Au film, maintained a nearly unchanged impedance, fluctuating within a narrow range of only 5.3%. This included readings of 103.25±6.21 Ω cm² on day 0 to 102.78±10.63 Ω cm² on day 28, and 105.15±8.28 Ω cm² on day 0 to 109.77±14.17 Ω cm² on day 28, respectively (n=5 for each group). On the other hand, the control device coated with Ag nanowires 16 experienced a significant impedance increase, from 250.53±15.88 Ω cm² on day 0 to 2,150.98±155.14 Ω cm² on day 28. Additionally, the electrochemical impedance of the sensing platform 10 (|Z|=105.1±4.4 Ω cm²) remained similar to that of the control device coated with the Au film (97.3±5.8 Ω cm²) at 1 kHz in PBS. The phase shift data suggested that both the sensing platform 10 and the control device coated with the Au film behaved like parallel resistor-capacitor (RC) circuits. The cyclic voltammetry analysis suggested that the sensing platform 10 demonstrated a total charge storage capacity of 0.51±0.33 mC cm², significantly higher than that (0.04±0.10 mC cm⁻²) of the control device coated with Ag nanowires 16, but slightly lower than that (0.66±0.39 mC cm⁻²) of the control device coated with the Au film (n=5 for each group). Overall, the sensing platform 10 exhibited electrical characteristics comparable to those of previously reported platforms using Au nanowires 16 as electrodes.

To ensure long-term reliable intracellular recording, the sensing platform 10 is required to provide high cell viability. To this end, the Ag nanowires 16 that were used in the experimental sensing platform 10 were fully covered with a thin layer of Au to prevent the release of toxic Ag ions into the surrounding medium. FIG. 2F displays the cell viability results of the sensing platform 10 with HL-1 cells at 24, 48, and 72 hours, as determined by an MTT assay (3-(4,5-dimethylthiazol-2-yl)-2,5-diphenyltetrazolium bromide), when the length of the exposed tip 12A was maintained around 5 μm. The cell viability of the sensing platform 10 remained consistently above 99% throughout the assay period, with no significant differences observed between groups (n=5 for each group). Generally, the diameter and height of the needle 12 and the density of the array correlated positively with the penetration force applied to the cell, affecting cell viability. The density of the needle array, as well as cell viability, is related to the interface tightness and stress between the cell and the sensing platform 10, which in turn influence penetration behavior and the quality of the recorded electrical signal. Previous research has shown successful cell tests with micro-sized needles with diameters ranging from 80 nm to 3 μm and heights ranging from 700 nm to 70 μm, and therefore such ranges are within the scope of the present invention. No significant differences in cell viability associated with changes in the diameters and lengths of the needles 12 were observed. The excellent cell viability of the sensing platform 10 after 72 hours of culture was also confirmed using a Calcein AM assay. Furthermore, confocal microscope images revealed that the sensing platform 10 supported HL-1 growth, with the cells reaching confluency and exhibiting beating activity.

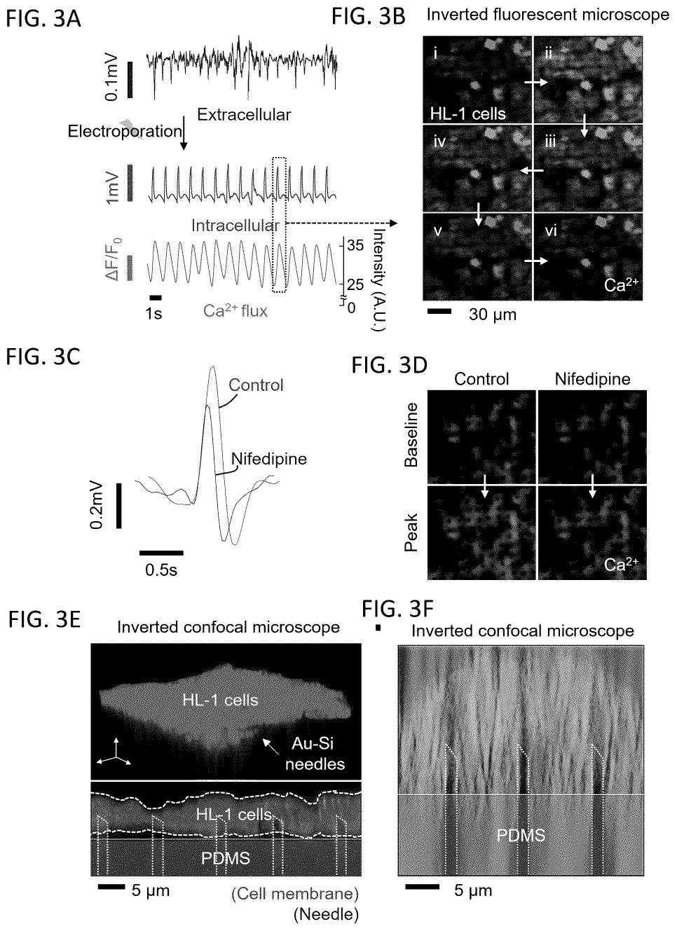

To demonstrate the capability of the sensing platform 10 in simultaneous intracellular recording and imaging, HL-1 mouse cardiomyocytes were cultured until they reached confluency and exhibited spontaneous beating. The surface of the sensing platform 10 was coated with a gelatin solution to ensure cell adhesion, and no additional functionalization agents were used. The HL-1 cells were cultured for 3-5 days until they achieved confluency and exhibited spontaneous beating. Initially, extracellular action potentials with amplitudes less than 0.1 mV were detected (FIG. 3A, top panel). To further improve the adhesion of the needles 12 to the cell membrane, mechanoporation was conducted by centrifuging at 500 rpm for one minute. Electroporation was followed using a series of biphasic square pulses to facilitate electrical access to the intracellular region. The beating interval remained at the same level before and after mechanoporation and electroporation, with the amplitude increasing drastically by 10 times up to 1 mV and a significantly reduced noise level (FIG. 3A, middle panel). In order to showcase the live imaging capabilities through the optical transparency of the sensing platform 10, concurrent calcium ion (Ca²⁺) flux imaging was simultaneously performed using an inverted microscope—a standard tool that is compatible only with transparent substrates. For Ca²⁺ flux imaging, Fluo-8, a fluorescent calcium indicator, was loaded into the cells for live imaging, and the alteration in fluorescent intensity (AF) was measured and compared with the resting intensity (F₀). The Ca²⁺ flux imaging is a well-established technique used to monitor the activity of electrogenic cells by tracking the changes in calcium flux evoked by action potentials. FIG. 3A (bottom panel) depicts the measured change in Ca²⁺ flux, demonstrating synchronized beating with electrical measurement at a frequency of 1 Hz.

FIG. 3B presents inverted fluorescent microscope images taken during a single action potential, showing a distinct peak of calcium ions upon depolarization through the opening of L-type calcium channels, enabling Ca²⁺ to enter the cell down its concentration gradient, followed by subsequent recovery through repolarization by the closing of the calcium channel. To evaluate the signal quality of the sensing platform 10, a comparative analysis of signal amplitude was conducted in comparison to a control group including the needles on a Si wafer. The measurements were limited to the cells that had been beating at approximately 1 Hz for at least one day, as the recorded amplitude and beating interval can vary with cell culture age. The action potentials of the cells were recorded using the sensing platform 10 (n=25), yielding an average amplitude of 1.30±0.43 mV. Under the same conditions, the intracellular action potentials of the cells were recorded using the control group (n=25), yielding an average amplitude of 1.29±0.31 mV. The measured peak amplitudes were not significantly different. These findings indicated that the sensing platform 10 offers similar capabilities for intracellular recording as the control group, while also providing mechanical compliance and optical transparency allowing for both reliable recording and simultaneous imaging.

To further demonstrate the utility of the sensing platform 10 in drug screening, 100 nM of an ion channel blocking drug nifedipine was administered to the cells to observe changes in action potential morphologies. Nifedipine is known to block L-type Ca²⁺ channels, thereby hindering the influx of Ca ions into the intracellular region, resulting in early repolarization and a reduction in peak amplitude. FIG. 3C shows that the amplitude was lowered, and the action potential duration was reduced compared to the control signal without administrating the drug. Specifically, the action potential duration at 50% of repolarization (APD50) decreased significantly from 120.05±13.02 ms to 104.80±7.42 ms, while the peak amplitude showed a significant decrease from 0.85±0.17 mV to 0.55±0.12 mV. Additionally, Ca²⁺ flux imaging showed that intensity decreased at the peak level after nifedipine treatment from 2.8 to 2.4 ΔF/F₀, aligning with the intracellular recording data (FIG. 3D), representing composite signal from multiple cells. Both the electrical and optical measurements of the change in action potential aligned with previous studies on nifedipine, highlighting the potential use of the sensing platform 10 in drug screening.

The ability of the needles 12 to penetrate a cell membrane and provide intracellular access was confirmed using fluorescent labeling with an inverted confocal microscope (FIG. 3E). The 3D and cross-sectional images, labeled with the cell membrane (in red) and needles (in blue), demonstrated the tight interface formed between the needles 12 and the cell membrane. The distance between the HL-1 cells and the PDMS electrically-insulating layer 30 was evaluated, resulting in an average of 0.13 μm with a standard deviation of 0.073 μm, through the analysis of the confocal image representing a signal-to-noise ratio of −4.6 dB. FIG. 3F shows a representative merged image obtained from both DIC and confocal microscope, showing the overlapped configuration of needles 12 and nucleus. The penetration of the needles 12 all the way through the cell cytoplasm can be confirmed on the combined basis of amplitude increase in electrical signal and cross-sectional confocal images.

Significant strides have been made toward developing 3D organ-like structures. These miniaturized organs mimic the multicellular composition, anatomy, and functionality of actual organs, providing a scaffold for investigating organ growth, maintenance, regeneration, and disease processes. Furthermore, these structures expedite more accurate drug screening and toxicity testing, reducing costs and development times for new pharmaceuticals and healthcare products. Utilizing various biosensors allows for real-time, minimally invasive monitoring of inter-cellular communication, revealing the influence of biochemical and biophysical environments on organ functionality. Investigations leading to the present invention evidenced the potential utility of the sensing platform 10 for simultaneous intracellular recording and imaging in a 3D-engineered cardiovascular tissue. The intracellular function was assessed via field potential measurements during Ca²⁺ flux imaging.

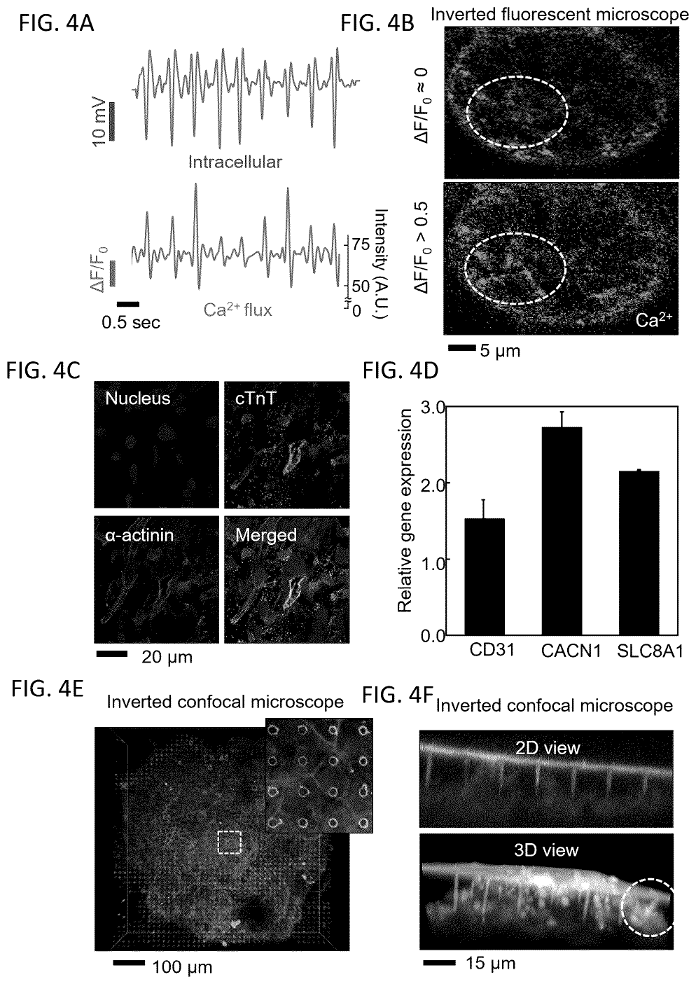

FIG. 4A presents measurement results of an intraorganoid field potential (top panel) and Ca²⁺ flux signal (bottom panel), with their clear synchronization observed every 0.5 seconds. Simultaneous imaging with an inverted fluorescence microscope confirmed that the Ca²⁺ flux signal from the 3D-engineered cardiac tissue consistently surpassed 0.5 ΔF/F₀ (FIG. 4B). The structural differences between 2D monolayered cells and 3D organoid cultures resulted in variations in the recorded signal. This is in addition to the differences stemming from the distinct measured parameters: intracellular action potential (FIG. 3A) and intraorganoid field potential (FIG. 4A). After 14 days of differentiation, the 3D-engineered tissue expressed α-actinin and cardiac troponin T (cTnT). The organoid displayed a cardiac sarcomeric actin structure, including Z-disc and cTnT (FIG. 4C), and exhibited increased gene expression of CD31, CACNAlC, and SLC8A1 compared to undifferentiated embryonic bodies (EBs) (FIG. 4D), signifying successful cardiovascular differentiation. Disparities observed in the relationship between electrical and fluorescence recordings, shown in FIGS. 3A-3F and 4A-4F, was attributed to the inherent differences in cell structures and the distinct recording methods employed for each.

FIG. 4E presents a representative confocal microscope image of the sensing platform 10 interfacing with the 3D cardiovascular tissue on top, stained with actin (in red) and nucleus (in blue), captured through the optically transparent substrate 14. The inset image, acquired at a single focal plane, emphasizes the presence of the needles 12 within the 3D cardiovascular tissue. The needles 12 were placed into the 3D tissue via gravity after a resting period of approximately 5 minutes, as confirmed by the side view of the inverted confocal microscope images in both 2D and 3D views (FIG. 4F). Notably, the needles 12 on the edge (white dotted circle) buckled due to its interaction with the non-linear surface of the 3D tissue, without fracturing or delaminating, owing to the mechanical compliance of the sensing platform 10 in accommodating mechanical discrepancies.

In view of the foregoing, the investigations evidenced the successful fabrication and testing of an optically transparent intracellular sensing platform 10 that combines vertically ordered Si needles 12 with a percolated network of Au—Ag nanowires 16 on an optically transparent flexible substrate 14. The sensing platform 10 offers low electrochemical impedance, high optical transparency, excellent mechanical compliance, and promotes cell viability, making it ideal for simultaneous intracellular recording and imaging. The sensing platform 10 can monitor intracellular electrophysiological functions over time, including cardiomyocytes and 3D-engineered cardiovascular tissue, and capture their functional changes upon the administration of a therapeutic drug. Moreover, this sensing platform 10 exhibits potential benefits for optogenetics, allowing light signals to traverse from both sides with notable efficiency, indicating a promising avenue for future research. This feature would not only support the investigation of cellular processes and responses to various stimuli, but also enhance drug development, personalized medicine approaches, and the study of complex cellular interactions using light-based modulation techniques.

As previously noted above, though the foregoing detailed description describes certain aspects of one or more particular embodiments of the invention and investigations associated with the invention, alternatives could be adopted by one skilled in the art. For example, sensing platform 10 could differ in appearance and construction from the embodiments described herein and shown in the drawings, process parameters could be modified, and appropriate materials could be substituted for those noted. As such, and again as was previously noted, it should be understood that the invention is not necessarily limited to any particular embodiment described herein or illustrated in the drawings.

1. A sensing platform comprising:
a flexible substrate;
micro-sized needles extending from a surface of the flexible substrate;
a percolated network of electrically-conductive nanowires on the surface of the flexible substrate; and
an electrically-insulating layer disposed on the surface of the flexible substrate and covering the percolated network of electrically-conductive nanowires so that only tips of the micro-sized needles protrude from and are exposed by the electrically-insulating layer.

2. The sensing platform of claim 1, wherein the flexible substrate is formed by an elastomer material.

3. The sensing platform of claim 2, wherein the electrically-insulating layer is formed by the elastomer material.

4. The sensing platform of claim 1, wherein the micro-sized needles are formed of silicon.

5. The sensing platform of claim 1, wherein the flexible substrate is optically transparent.

6. The sensing platform of claim 1, wherein the micro-sized needles have heights of about 700 nm to about 700 micrometers.

7. The sensing platform of claim 1, wherein the micro-sized needles have diameters of about 80 nm to about 3 micrometers.

8. The sensing platform of claim 1, wherein tips of the micro-sized needles have lengths of about 2 micrometers to about 25 micrometers.

9. The sensing platform of claim 1, the sensing platform further comprising an optically transparent conduction path leading from the flexible substrate and comprising a portion of the percolated network of nanowires.

10. The sensing platform of claim 1, the sensing platform further comprising a cell culture well affixed to the electrically-insulating layer and surrounding the tips of the micro-sized needles.

11. A method of using the sensing platform of claim 10, the method comprising:
depositing a composition within the cell culture well so as to contact and be pierced by the tips of the micro-sized needles extend; and then
causing the sensing platform to electrically interact with the composition.

12. The method of claim 11, wherein the composition is a cell culture.

13. The method of claim 12, wherein the electrical interaction comprises simultaneous intracellular recording of the cell culture.

14. The method of claim 12, wherein the electrical interaction comprises simultaneous intracellular recording and imaging of the cell culture.

15. A method of fabricating the sensing platform of claim 1, the method comprising:
providing a first substrate having pillars extending from a surface thereof;
locally reducing diameters of the pillars at locations thereof adjacent the first substrate;
embedding distal ends of the pillars in the flexible substrate;
expanding the flexible substrate to cause the pillars to fracture at the locations thereof adjacent the first substrate and detach therefrom to define the micro-sized needles extending from the flexible substrate;
depositing the percolated network of electrically-conductive nanowires on the surface of the flexible substrate; and then
depositing the electrically-insulating layer on the surface of the flexible substrate so as to cover the percolated network of electrically-conductive nanowires and so that only the tips of the micro-sized needles protrude from and are exposed by the electrically-insulating layer.
